(19) United States
(12) Patent Application Publication
(10) Pub. No.: US 2003/0100548 A1
(43) Pub. Date: May 29, 2003

(54) Arylpiperazines and their use as metallaproteinase inhibiting agents (mmp)

(76) Inventors: Bernard Christophe Barlaam, Reims (FR); Nicholas John Newcombe, Cheshire (GB); Howard Tucker, Cheshire (GB); David Waterson, Cheshire (GB)

Correspondence Address: Janis K Fraser, Fish & Richardson, 225 Franklin Street, Boston, MA, 02110-2804, US

(21) Appl. No.: 10/204,368

(22) PCT Filed: Feb. 15, 2001
(86) PCT No.: PCT/GB01/00613

(30) Foreign Application Priority Data
Feb. 21, 2000 (EP) 00400468.5

(51) Int. Cl. C07D 417/02; C 07D 4 3/02; C07D 413/02; C 07D 4 1/02; A61K 31/551; A61K 31/55; A61K 31/541; A61K 31/5377; A61K 31/496; A61K 31/4545; A61K 31/454; A61K 31/4025

(52) U.S. Cl. 514/217.04; 514/217.05; 514/217.09; 514/218; 514/227.8; 514/235.8; 514/252.13; 514/326; 514/254.01; 514/422; 544/60; 540/575; 540/360; 544/129; 544/120

(57) ABSTRACT

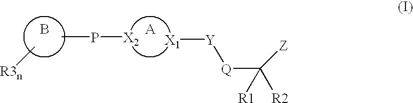

Compounds of formula (I) wherein Z is —CH2SR and R represents hydrogen or —COCH₃, are useful as metalloproteinase inhibitors, especially as inhibitors of MMP 13.

The present invention relates to compounds useful in the inhibition of metalloproteinases and in particular to pharmaceutical compositions comprising these, as well as their use.

The compounds of this invention are inhibitors of one or more metalloproteinase enzymes. Metalloproteinases are a superfamily of proteinases (enzymes) whose numbers in recent years have increased dramatically. Based on structural and functional considerations these enzymes have been classified into families and subfamilies as described in N. M. Hooper (1994) FEBS Letters 354:1-6. Examples of metalloproteinases include the matrix metalloproteinases (MMP) such as the collagenases (MMP1, MMP8, MMP13), the gelatinases (MMP2, MMP9), the stromelysins (MMP3, MMP10, MMP11), matrilysin (MMP7), metalloelastase (MMP12), enamelysin (MMP19), the MT-MMPs (MMP14, MMP15, MMP16, MMP17); the reprolysin or adamalysin or MDC family which includes the secretases and sheddases such as TNF converting enzymes (ADAM10 and TACE); the astacin family which include enzymes such as procollagen processing proteinase (PCP); and other metalloproteinases such as aggrecanase, the endothelin converting enzyme family and the angiotensin converting enzyme family.

Metalloproteinases are believed to be important in a plethora of physiological disease processes that involve tissue remodelling such as embryonic development, bone formation and uterine remodelling during menstruation. This is based on the ability of the metalloproteinases to cleave a broad range of matrix substrates such as collagen, proteoglycan and fibronectin. Metalloproteinases are also believed to be important in the processing, or secretion, of biological important cell mediators, such as tumour necrosis factor (TNF); and the post translational proteolysis processing, or shedding, of biologically important membrane proteins, such as the low affinity IgE receptor CD23 (for a more complete list see N. M. Hooper et al., (1997) Biochem J. 321:265-279).

Metalloproteinases have been associated with many disease conditions. Inhibition of the activity of one or more metalloproteinases may well be of benefit in these disease conditions, for example: various inflammatory and allergic diseases such as, inflammation of the joint (especially rheumatoid arthritis, osteoarthritis and gout), inflammation of the gastro-intestinal tract (especially inflammatory bowel disease, ulcerative colitis and gastritis), inflammation of the skin (especially psoriasis, eczema, dermatitis); in tumour metastasis or invasion; in disease associated with uncontrolled degradation of the extracellular matrix such as osteoarthritis; in bone resorptive disease (such as osteoporosis and Paget's disease); in diseases associated with aberrant angiogenesis; the enhanced collagen remodelling associated with diabetes, periodontal disease (such as gingivitis), corneal ulceration, ulceration of the skin, post-operative conditions (such as colonic anastomosis) and dermal wound healing; demyelinating diseases of the central and peripheral nervous systems (such as multiple sclerosis); Alzheimer's disease; extracellular matrix remodelling observed in cardiovascular diseases such as restenosis and atheroscelerosis; and chronic obstructive pulmonary diseases, COPD (for example, the role of MMPs such as MMP12 is discussed in Anderson & Shinagawa, 1999, Current Opinion in Anti-inflammatory and Immunomodulatory Investigational Drugs, 1(1): 29-38).

A number of metalloproteinase inhibitors are known; different classes of compounds may have different degrees of potency and selectivity for inhibiting various metalloproteinases. We have discovered a new class of compounds that are inhibitors of metalloproteinases and are of particular interest in inhibiting MMP-13, as well as MMP-9. The compounds of this invention have beneficial potency and/or pharmacokinetic properties.

MMP13, or collagenase 3, was initially cloned from a cDNA library derived from a breast tumour [J. M. P. Freije et al. (1994) Journal of Biological Chemistry 269(24):16766-16773]. PCR-RNA analysis of RNAs from a wide range of tissues indicated that MMP13 expression was limited to breast carcinomas as it was not found in breast fibroadenomas, normal or resting mammary gland, placenta, liver, ovary, uterus, prostate or parotid gland or in breast cancer cell lines (T47-D. MCF-7 and ZR75-1). Subsequent to this observation MMP13 has been detected in transformed epidermal keratinocytes [N. Johansson et al., (1997) Cell Growth Differ. 8(2):243-250], squamous cell carcinomas [N. Johansson et al., (1997) Am. J. Pathol. 151(2):499-508] and epidermal tumours [K. Airola et al., (1997) J. Invest. Dermatol. 109(2):225-231]. These results are suggestive that MMP13 is secreted by transformed epithelial cells and may be involved in the extracellular matrix degradation and cell-matrix interaction associated with metastasis especially as observed in invasive breast cancer lesions and in malignant epithelia growth in skin carcinogenesis.

Recent published data implies that MMP13 plays a role in the turnover of other connective tissues. For instance, consistent with MMP13's substrate specificity and preference for degrading type II collagen [P. G. Mitchell et al., (1996) J. Clin. Invest. 97(3):761-768; V. Knauper et al., (1996) The Biochermical Journal 271:1544-1550], MMP13 has been hypothesised to serve a role during primary ossification and skeletal remodelling [M. Stahle-Backdahl et al., (1997) Lab. Invest. 76(5):717-728; N. Johansson et al., (1997) Dev. Dyn. 208(3):387-397], in destructive joint diseases such as rheumatoid and osteo-arthritis [D. Wernicke et al., (1996) J. Rheumatol. 23:590-595; P. G. Mitchell et al., (1996) J. Clin. Invest. 97(3):761-768; O. Lindy et al., (1997) Arthritis Rheum 40(8):1391-1399]; and during the aseptic loosening of hip replacements [S. Imai et al., (1998) J. Bone Joint Surg. Br. 80(4):701-710]. MMP13 has also been implicated in chronic adult periodontitis as it has been localised to the epithelium of chronically inflamed mucosa human gingival tissue [V. J. Uitto et al., (1998) Am. J. Pathol 152(6):1489-1499] and in remodelling of the collagenous matrix in chronic wounds [M. Vaalamo et al., (1997) J. Invest. Dermatol. 109(1):96-101].

MMP9 (Gelatinase B; 92 kDa TypeIV Collagenase; 92 kDa Gelatinase) is a secreted protein which was first purified, then cloned and sequenced, in 1989 (S. M. Wilhelm et al (1989) J. Biol Chem. 264 (29): 17213-17221. Published erratum in J. Biol Chem. (1990) 265 (36): 22570.). A recent review of MMP9 provides an excellent source for detailed information and references on this protease: T. H. Vu & Z. Werb (1998) (In: Matrix Metalloproteinases. 1998. Edited by W. C. Parks & R. P. Mecham. pp115-148. Academic Press. ISBN 0-12-545090-7). The following points are drawn from that review by T. H. Vu & Z. Werb (1998).

The expression of MMP9 is restricted normally to a few cell types, including trophoblasts, osteoclasts, neutrophils and macrophages. However, it's expression can be induced in these same cells and in other cell types by several mediators, including exposure of the cells to growth factors or cytokines. These are the same mediators often implicated in initiating an inflammatory response. As with other secreted MMPs, MMP9 is released as an inactive Pro-enzyme which is subsequently cleaved to form the enzymatically active enzyme. The proteases required for this activation in vivo are not known. The balance of active MMP9 versus inactive enzyme is further regulated in vivo by interaction with TIMP-1 (Tissue Inhibitor of Metalloproteinases-1), a naturally-occurring protein. TIMP-1 binds to the C-terminal region of MMP9, leading to inhibition of the catalytic domain of MMP9. The balance of induced expression of ProMMP9, cleavage of Pro- to active MMP9 and the presence of TIMP-1 combine to determine the amount of catalytically active MMP9 which is present at a local site. Proteolytically active MMP9 attacks substrates which include gelatin, elastin, and native Type IV and Type V collagens. it has no activity against native Type I collagen, proteoglycans or laminins.

There has been a growing body of data implicating roles for MMP9 in various physiological and pathological processes. Physiological roles include the invasion of embryonic trophoblasts through the uterine epithelium in the early stages of embryonic implantation; some role in the growth and development of bones; and migration of inflammatory cells from the vasculature into tissues. Increased MMP9 expression has observed in certain pathological conditions, thereby implicating MMP9 in disease processed such as arthritis, tumour metastasis, Alzheimer's, Multiple Sclerosis, and plaque rupture in atherosclerosis leading to acute coronary conditions such as Myocardial Infarction.

We have now discovered compounds that are potent MMP13 inhibitors and have desirable activity profiles.

In a first aspect of the invention we provide compounds of the formula I

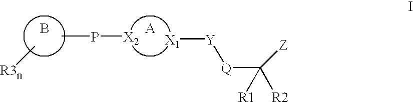

wherein ring B is a monocyclic or bicyclic alkyl, aryl, aralkyl, heteroaryl or heteroaralkyl ring comprising up to 12 ring atoms and containing one or more heteroatoms independently chosen from N, O, and S; alternatively ring B may be biphenyl; ring B may optionally be linked to ring A by a C1-4 alkyl or a C1-4 alkoxy chain linking the 2-position of ring B with a carbon atom alpha to X2;

each R3 is independently selected from hydrogen, halogen, NO2, COOR wherein R is hydrogen or C1-6alkyl, CN, CF3, C1-6 alkyl, —S—C1-6 alkyl, —SO—C1-6 alkyl, —SO2-C1-6 alkyl, C1-6 alkoxy and up to C10 aryloxy, n is 1,2 or 3;

P is —(CH₂)n- wherein n=0, 1, 2, or P is an alkene or alkyne chain of up to six carbon atoms; where X2 is C, P may be -Het-, —(CH[R6])n-Het-, -Het-(CH[R6]n-or -Het-(CH[R6])n-Het-, wherein Het is selected from —CO—, —S—, SO—, —SO2-, —NR6-, or —O— wherein n is 1 or 2, or P may be selected from —CO—N(R6)-, —N(R6)-CO—, —SO2-N(R6)- and —N(R6)-SO2-, and R6 is hydrogen, C1-6 alkyl, up to C10 aralkyl or up to C9 heteroalkyl;

ring A is a 5-7 membered aliphatic ring and may optionally be mono- or di-substituted by optionally substituted C1-6 alkyl or C1-6 alkoxy, each substituent being independently selected from halogen, C1-6 alkyl or an oxo group;

X1 and X2 are independently selected from N and C, where a ring substituent on ring A is an oxo group this is preferably adjacent a ring nitrogen atom;

Y is selected from —SO2- and —CO—;

Q is selected from —C(R7)(R8)-, —C(R7)(R8)-CH2-, —N(R7)-, and —N(R7)-CH2- wherein R7 is hydrogen, C1-6 alkyl, up to C10 aralkyl, up to C9 heteroalkyl, up to C10 aryl, up to C9 heteroaryl, and R8 is H, C 1-6 alkyl, or together with R7 forms a carbocyclic or heterocyclic spiro 5, 6 or 7 membered ring, the latter containing at least one heteroatom selected from N, O, and S;

R1 is H, C1-6 alkyl, C5-7 cycloalkyl, up to C10aryl, up to C10heteroaryl, up to C12aralkyl, or up to C12heteroarylalkyl, all optionally substituted by up to three groups independently selected from NO2, CF3, halogen, C1-4alkyl, carboxy(C1-4)alkyl, up to C6cycloalkyl, —OR4, —SR4, C1-4alkyl substituted with —OR4, SR4 (and its oxidised analogues), NR4, N—Y—R4, or C1-4alkyl-Y—NR4;

R4 is hydrogen, C1-6 alkyl, up to C10 aryl or up to C10 heteroaryl or up to C9 aralkyl, each independently optionally substituted by halogen, NO2, CN, CF3, C1-6 alkyl, —S—C1-6 alkyl, —SO—C1-6 alkyl, —SO2-C1-6 alkyl or C1-6 alkoxy;

R2 is H, C1-6 alkyl, or together with R1 forms a carbocyclic or heterocyclic spiro 5, 6 or 7 membered ring, the latter containing at least one heteroatom selected from N, O, and S;

also the group Q can be linked to either R1 or R2 to form a 5, 6 or 7 membered alkyl or heteroalkyl ring comprising one or more of O, S and N; and

Z is —CH₂—SR wherein R is hydrogen or COCH₃

Any alkyl groups outlined above may be straight chain or branched.

Convenient values for the above groups include the following:

ring A=a 5-6 membered aliphatic ring, such as a piperazine ring, and may optionally be mono- or di-substituted by optionally substituted C1-6 alkyl or C1-6 alkoxy, each substituent being independently selected from halogen, C1-6 alkyl or an oxo group;

R3=hydrogen, halogen, NO2, CF3, C1-4 alkyl, or C1-4 alkoxy, n is 1 or 2, such as individually 4-fluoro, CF3, 4-chloro and 3,4-dichloro;

ring B=monocyclic or bicyclic aryl, aralkyl or heteroaryl having up to 10 ring atoms, especially monocyclic aryl, aralkyl or heteroaryl having up to 7 ring atoms, more especially monocyclic aryl or heteroaryl having up to 6 ring atoms, such as a phenyl or pyridyl ring;

P=—(CH2)n- wherein n is 0 or 1, or —O—, or —CO—N(R6)-;

one or both of X2 and X1=N, or X1 is N, or X2 is C;

Y=—SO2-, Y=—CO—;

Q=—CH(R7)-, —CH(R7)-CH2-, or —N(R7)-CH2- wherein R7 is hydrogen or C1-6 alkyl; also where Q is linked to R1 or R2 to form a C5-7 alkyl or heteroalkyl ring such as a cyclohexyl ring;

R1=hydrogen, C1-6alkyl, C5-7 cycloalkyl, up to C12aralkyl, up to C11heteroarylalkyl, up to C10 aryl or heteroaryl such as up to C6 aryl; all optionally substituted by up to three halogen atoms, or by CF3;

R2=hydrogen, or together with R1 represent a carbocyclic or heterocyclic spiro 5- or 6 membered ring, such as a tetrahydropyran ring.

Particular values for the above groups include the following:

R3=hydrogen, halogen such as chlorine, bromine or fluorine, NO2, CF3, methyl, ethyl, methoxy, ethoxy, particularly methoxy or fluorine;

ring B=a monocyclic aryl, aralkyl or heteroaryl ring having up to 7 ring atoms such as phenyl, biphenyl, napthyl, pyridyl, pyrimidinyl, pyrazinyl and pyridazinyl, especially phenyl, pyridyl and pyrimidyl, more especially phenyl, 2-pyridyl and 2,4-pyrimidyl;

P=a direct bond;

both X2 and X1 are N;

Y=—SO2-;

Q=—CH2-;

R1 is phenyl, 4-trifluoromethylphenyl, phenethyl, phenpropyl, isobutyl, cyclopentyl, benzyloxymethyl, 3,4-dichlorophenyl, pyridyl, pyridylethyl, thiophenylpropyl, bromothiophenyl, pyrimidinylethyl, pyrimidinylpropyl, pyridylethyl, pyridylpropyl or together with R2 is spirocyclohexane or spiro-4-pyran;

R2 is hydrogen.

Further convenient values include R3 being halogen, the substituent is preferably meta or para to the ring junction where ring B is an aryl or heteroaryl ring, where ring B is phenyl then especially 4-fluoro and where ring B is pyridyl then 3-, or 4-chloro (as appropriate);

Q=—CH2-.

Particular values for R4 include up to C10 aryl optionally substituted by halogen, NO2, CN, CF3, C1-6 alkyl, —S—C1-6 alkyl, —SO—C1-6 alkyl, —SO2-C1-6 alkyl or C1-6 alkoxy.

Particular combinations of Rings B and A include phenyl and piperazinyl; pyridyl and piperazinyl, and pyrimidine and piperazinyl respectively.

Particular alicyclic, fused and heterocyclic rings for ring B include any one of the following:

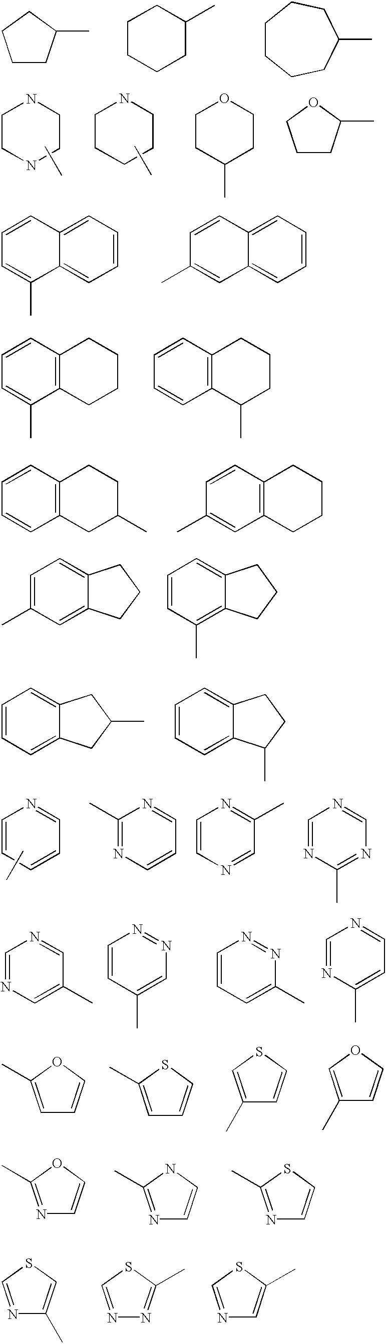

Particular rings for ring A include any one of the following:

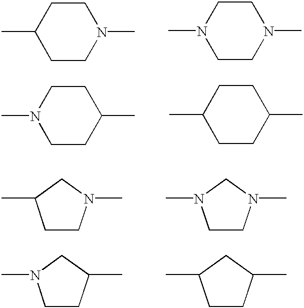

and its corresponding seven membered analogue(s).

It will be appreciated that the particular substituents and number of substituents on rings A and B are selected so as to avoid sterically undesirable combinations. This also applies to rings as may be formed by R1 and Q, R2 and Q as well as R7 and R8.

Each exemplified compound represents a particular and independent aspect of the invention.

Where optically active centres exist in the compounds of formula I, we disclose all individual optically active forms and combinations of these as individual specific embodiments of the invention, as well as their corresponding racemates. Racemates may be separated into individual optically active forms using known procedures (cf. Advanced Organic Chemistry: 3rd Edition: author J March, p104-107) including for example the formation of diastereomeric derivatives having convenient optically active auxiliary species followed by separation and then cleavage of the auxiliary species.

It will be appreciated that the compounds according to the invention may contain one or more asymmetrically substituted carbon atoms. The presence of one or more of these asymmetric centres (chiral centres) in a compound of formula I can give rise to stereoisomers, and in each case the invention is to be understood to extend to all such stereoisomers, including enantiomers and diastereomers, and mixtures including racemic mixtures thereof.

Where tautomers exist in the compounds of formula I, we disclose all individual tautomeric forms and combinations of these as individual specific embodiments of the invention.

As previously outlined the compounds of the invention are metalloproteinase inhibitors, in particular they are inhibitors of MMP13. Each of the above indications for the compounds of the formula I represents an independent and particular embodiment of the invention. Whilst we do not wish to be bound by theoretical considerations, the compounds of the invention are believed to show selective inhibition for any one of the above indications relative to any MMP1 inhibitory activity, by way of non-limiting example they may show 100-1000 fold selectivity over any MMP1 inhibitory activity.

Certain compounds of the invention are of particular use as aggrecanase inhibitors ie. inhibitors of aggrecan degradation. Certain compounds of the invention are of particular use as inhibitors of MMP9 and/or MMP12.

The compounds of the invention may be provided as pharmaceutically acceptable salts. These include acid addition salts such as hydrochloride, hydrobromide, citrate and maleate salts and salts formed with phosphoric and sulphuric acid. In another aspect suitable salts are base salts such as an alkali metal salt for example sodium or potassium, an alkaline earth metal salt for example calcium or magnesium, or organic amine salt for example triethylamine.

They may also be provided as in vivo hydrolysable esters. These are pharmaceutically acceptable esters that hydrolyse in the human body to produce the parent compound. Such esters can be identified by administering, for example intravenously to a test animal, the compound under test and subsequently examining the test animal's body fluids. Suitable in vivo hydrolysable esters for carboxy include methoxymethyl and for hydroxy include formyl and acetyl, especially acetyl.

In order to use a compound of the formula I or a pharmaceutically acceptable salt or in vivo hydrolysable ester thereof for the therapeutic treatment (including prophylactic treatment) of mammals including humans, it is normally formulated in accordance with standard pharmaceutical practice as a pharmaceutical composition.

Therefore in another aspect the present invention provides a pharmaceutical composition which comprises a compound of the formula I or a pharmaceutically acceptable salt or an in vivo hydrolysable ester and pharmaceutically acceptable carrier.

The pharmaceutical compositions of this invention may be administered in standard manner for the disease condition that it is desired to treat, for example by oral, topical, parenteral, buccal, nasal, vaginal or rectal adminstration or by inhalation. For these purposes the compounds of this invention may be formulated by means known in the art into the form of, for example, tablets, capsules, aqueous or oily solutions, suspensions, emulsions, creams, ointments, gels, nasal sprays, suppositories, finely divided powders or aerosols for inhalation, and for parenteral use (including intravenous, intramuscular or infusion) sterile aqueous or oily solutions or suspensions or sterile emulsions.

In addition to the compounds of the present invention the pharmaceutical composition of this invention may also contain, or be co-administered (simultaneously or sequentially) with, one or more pharmacological agents of value in treating one or more disease conditions referred to hereinabove.

The pharmaceutical compositions of this invention will normally be administered to humans so that, for example, a daily dose of 0.5 to 75 mg/kg body weight (and preferably of 0.5 to 30 mg/kg body weight) is received. This daily dose may be given in divided doses as necessary, the precise amount of the compound received and the route of administration depending on the weight, age and sex of the patient being treated and on the particular disease condition being treated according to principles known in the art.

Typically unit dosage forms will contain about 1 mg to 500 mg of a compound of this invention.

Therefore in a further aspect, the present invention provides a compound of the formula I or a pharmaceutically acceptable salt or in vivo hydrolysable ester thereof for use in a method of therapeutic treatment of the human or animal body. In particular we disclose use in the treatment of a disease or condition mediated by MMP13 and/or aggrecanase and/or MMP9 and/or MMP12.

In yet a further aspect the present invention provides a method of treating a metalloproteinase mediated disease condition which comprises administering to a warm-blooded animal a therapeutically effective amount of a compound of the formula I or a pharmaceutically acceptable salt or in vivo hydrolysable ester thereof. Metalloproteinase mediated disease conditions include arthritis (such as osteoarthritis), atherosclerosis, chronic obstructive pulmonary diseases (COPD).

In another aspect the present invention provides a process for preparing a compound of the formula I or a pharmaceutically acceptable salt or in vivo hydrolysable ester thereof which process comprises

(a) reacting a compound of the formula II with a compound of the formula III

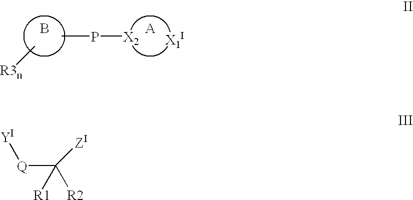

wherein X₁^{l} represents X₁ or a precursor of X₁ (whether by modification or displacement) or an activated form of X₁ suitable for reaction with Y^{l};

Y^{l} represents Y, a precursor of Y, or an activated form of Y suitable for reaction with X₁^{l}; by way of non-limiting example, when X₁ is C then X₁ may be derivatised to include a precursor of Y for reaction with a compound of formula III wherein Y^{l} is a precursor of Y;

Z^{l} represents an acid or ester group or a protected aldehyde, following reaction of II and III this is converted to a group —CH₂X wherein X represents a leaving group, this in turn is reacted with an appropriate sulphur reagent such as e.g., metal hydrosulphides, thiourea or thiolacetates to yield a group Z (as defined); and optionally thereafter forming a pharmaceutically acceptable salt or in vivo hydrolysable ester of the compound of formula I;

or

b) reacting a compound of the formula IV with a compound of the formula V

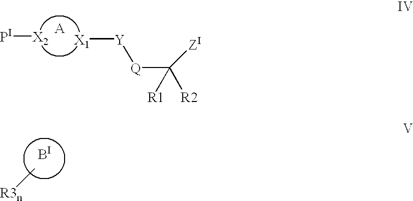

wherein B^{l} represents a suitable ring function or substituent group for reaction with P¹;

Z^{l} is a protected thiol group; and

P^{l} represents a suitably activated form of the linker P for reaction with B^{l} or where X2=N then P^{l} may be present on ring A rather than ring B or, as required, the linker P may be formed by appropriate reaction of precursor groups P″ and P′″ provided on rings B^{l} and A respectively, or vice versa;

and deprotecting the group Z^{l} to yield a group Z (as defined);

and optionally thereafter forming a pharmaceutically acceptable salt or in vivo hydrolysable ester of the compound of formula I.

A compound of the formula II is conveniently prepared by reacting a compound of the formula VI with a compound of the formula VII

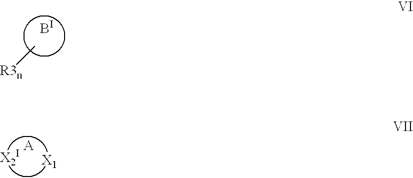

wherein B^{l} represents a suitable ring function or substituent group, X₂^{l} represents X₂ or a precursor of X₂ (whether by modification or displacement) or an activated form of X₂ suitable for reaction with B^{l} and wherein B^{l} and X₂^{l} when reacted together provide the linker P between ring A and ring B in the compound of formula II. By way of non-limiting example, when X₂ is N then ring B is suitably derivatised to introduce the linker P via B^{l}, and when X₂ is C then both ring B and ring A are suitably derivatised to provide the linker P by the reaction of B^{l} and X₂^{l}.

It will be appreciated that many of the relevant starting materials are commercially available.

The compounds of the invention may be evaluated for example in the following assays:

Isolated Enzyme Assays

Matrix Metalloproteinase Family Including for Example MMP13.

Recombinant human proMMP13 may be expressed and purified as described by Knauper et al. [V. Knauper et al., (1996) The Biochemical Journal 271:1544-1550 (1996)]. The purified enzyme can be used to monitor inhibitors of activity as follows: purified proMMP13 is activated using 1 mM amino phenyl mercuric acid (APMA), 20 hours at 21° C.; the activated MMP13 (11.25 ng per assay) is incubated for 4-5 hours at 35° C. in assay buffer (0.1M Tris-HCl, pH 7.5 containing 0.1M NaCl, 20 mM CaCl2, 0.02 mM ZnCl and 0.05% (w/v) Brij 35 using the synthetic substrate 7-methoxycoumarin-4-yl)acetyl.Pro.Leu.Gly.Leu.N-3-(2,4-dinitrophenyl)-L-2,3-diaminopropionyl.Ala.Arg.NH₂ in the presence or absence of inhibitors. Activity is determined by measuring the fluorescence at λex 328 nm and λem 393 nm. Percent inhibition is calculated as follows: % Inhibition is equal to the [Fluorescence_{plus inhibitor}-Fluorescence_{background}] divided by the [Fluorescence_{minus inhibitor}-Fluorescence_{background}].

A similar protocol can be used for other expressed and purified pro MMPs using substrates and buffers conditions optimal for the particular MMP, for instance as described in C. Graham Knight et al., (1992) FEBS Lett. 296(3):263-266.

Adamalysin Family Including for Example TNF Convertase

The ability of the compounds to inhibit proTNFα convertase enzyme may be assessed using a partially purified, isolated enzyme assay, the enzyme being obtained from the membranes of THP-1 as described by K. M. Mohler et al., (1994) Nature 370:218-220. The purified enzyme activity and inhibition thereof is determined by incubating the partially purified enzyme in the presence or absence of test compounds using the substrate 4′,5′-Dimethoxy-fluoresceinyl Ser.Pro.Leu.Ala.Gln.Ala.Val.Arg.Ser.Ser.Ser.Arg.Cys(4-(3-succinimid-1-yl)-fluorescein)-NH₂ in assay buffer (50 mM Tris HCl, pH 7.4 containing 0.1% (w/v) Triton X-100 and 2 mM CaCl₂), at 26° C. for 18 hours. The amount of inhibition is determined as for MMP13 except λex 490 nm and λem 530 nm were used. The substrate was synthesised as follows. The peptidic part of the substrate was assembled on Fmoc-NH-Rink-MBHA-polystyrene resin either manually or on an automated peptide synthesiser by standard methods involving the use of Fmoc-amino acids and O-benzotriazol-1-yl-N,N,N′,N′-tetramethyluronium hexafluorophosphate (HBTU) as coupling agent with at least a 4- or 5-fold excess of Fmoc-amino acid and HBTU. Ser¹ and Pro² were double-coupled. The following side chain protection strategy was employed; Ser¹(But), Gln⁵(Trityl), Arg^{8,12}(Pmc or Pbf), Ser^{9,10,11}(Trityl), Cys¹³(Trityl). Following assembly, the N-terminal Fmoc-protecting group was removed by treating the Fmoc-peptidyl-resin with in DMF. The amino-peptidyl-resin so obtained was acylated by treatment for 1.5-2 hr at 70° C. with 1.5-2 equivalents of 4′,5′-dimethoxy-fluorescein4(5)-carboxylic acid [Khanna & Ullman, (1980) Anal Biochem. 108:156-161) which had been preactivated with diisopropylcarbodiimide and 1-hydroxybenzotriazole in DMF]. The dimethoxyfluoresceinyl-peptide was then simultaneously deprotected and cleaved from the resin by treatment with trifluoroacetic acid containing 5% each of water and triethylsilane. The dimethoxyfluoresceinyl-peptide was isolated by evaporation, trituration with diethyl ether and filtration. The isolated peptide was reacted with 4-(N-maleimido)-fluorescein in DMF containing diisopropylethylamine, the product purified by RP-HPLC and finally isolated by freeze-drying from aqueous acetic acid. The product was characterised by MALDI-TOF MS and amino acid analysis.

Natural Substrates

The activity of the compounds of the invention as inhibitors of aggrecan degradation may be assayed using methods for example based on the disclosures of E. C. Arner et al., (1998) Osteoarthritis and Cartilage 6:214-228; (1999) Journal of Biological Chemistry, 274 (10), 6594-6601 and the antibodies described therein. The potency of compounds to act as inhibitors against collagenases can be determined as described by T. Cawston and A. Barrett (1979) Anal. Biochem. 99:340-345.

Inhibition of Metalloproteinase Activity in Cell/Tissue Based Activity Test as an Agent to Inhibit Membrane Sheddases such as TNF Convertase

The ability of the compounds of this invention to inhibit the cellular processing of TNFα production may be assessed in THP-1 cells using an ELISA to detect released TNF essentially as described K. M. Mohler et al., (1994) Nature 370:218-220. In a similar fashion the processing or shedding of other membrane molecules such as those described in N. M. Hooper et al., (1997) Biochem. J. 321:265-279 may be tested using appropriate cell lines and with suitable antibodies to detect the shed protein.

Test as an Agent to Inhibit Cell Based Invasion

The ability of the compound of this invention to inhibit the migration of cells in an invasion assay may be determined as described in A. Albini et al., (1987) Cancer Research 47:3239-3245.

Test as an Agent to Inhibit Whole Blood TNF Sheddase Activity

The ability of the compounds of this invention to inhibit TNFα production is assessed in a human whole blood assay where LPS is used to stimulate the release of TNFα. Heparinized (10 Units/ml) human blood obtained from volunteers is diluted 1:5 with medium (RPMI1640+bicarbonate, penicillin, streptomycin and glutamine) and incubated (160 μl) with 20 μl of test compound (triplicates), in DMSO or appropriate vehicle, for 30 min at 37° C. in a humidified (5%CO₂/95%air) incubator, prior to addition of 20 μl LPS (E. coli. 0111:B4; final concentration 10 μg/ml). Each assay includes controls of diluted blood incubated with medium alone (6 wells/plate) or a known TNFα inhibitor as standard. The plates are then incubated for 6 hours at 37° C. (humidified incubator), centrifiged (2000 rpm for 10 min; 4° C.), plasma harvested (50-100 μl) and stored in 96 well plates at −70° C. before subsequent analysis for TNFα concentration by ELISA.

Test as an Agent to Inhibit in vitro Cartilage Degradation

The ability of the compounds of this invention to inhibit the degradation of the aggrecan or collagen components of cartilage can be assessed essentially as described by K. M. Bottomley et al., (1997) Biochem J. 323:483-488.

Pharmacodynamic Test

To evaluate the clearance properties and bioavailability of the compounds of this invention an ex vivo pharmacodynamic test is employed which utilises the synthetic substrate assays above or alternatively HPLC or Mass spectrometric analysis. This is a generic test which can be used to estimate the clearance rate of compounds across a range of species. Animals (e,g. rats, marmosets) are dosed iv or po with a soluble formulation of compound (such as 20% w/v DMSO, 60% w/v PEG400) and at subsequent time points (e.g. 5, 15, 30, 60, 120, 240, 480, 720, 1220 mins) the blood samples are taken from an appropriate vessel into 10U heparin. Plasma fractions are obtained following centrifugation and the plasma proteins precipitated with acetonitrile (80% w/v final concentration). After 30 mins at −20° C. the plasma proteins are sedimented by centrifugation and the supernatant fraction is evaporated to dryness using a Savant speed vac. The sediment is reconstituted in assay buffer and subsequently analysed for compound content using the synthetic substrate assay. Briefly, a compound concentration-response curve is constructed for the compound undergoing evaluation. Serial dilutions of the reconstituted plasma extracts are assessed for activity and the amount of compound present in the original plasma sample is calculated using the concentration-response curve taking into account the total plasma dilution factor.

In vivo Assessment

Test as an Anti-TNF Agent

The ability of the compounds of this invention as ex vivo TNFα inhibitors is assessed in the rat. Briefly, groups of male Wistar Alderley Park (AP) rats (180-210 g) are dosed with compound (6 rats) or drug vehicle (10 rats) by the appropriate route e.g. peroral (p.o.), intraperitoneal (i.p.), subcutaneous (s.c.). Ninety minutes later rats are sacrificed using a rising concentration of CO₂ and bled out via the posterior vena cavae into 5 Units of sodium heparin/ml blood. Blood samples are immediately placed on ice and centrifuged at 2000 rpm for 10 min at 4° C. and the harvested plasmas frozen at −20° C. for subsequent assay of their effect on TNFα production by LPS-stimulated human blood. The rat plasma samples are thawed and 175 μl of each sample are added to a set format pattern in a 96U well plate. Fifty μl of heparinized human blood is then added to each well, mixed and the plate is incubated for 30 min at 37° C. (humidified incubator). LPS (25 μl ; final concentration 10 μg/ml) is added to the wells and incubation continued for a further 5.5 hours. Control wells are incubated with 25 μl of medium alone. Plates are then centrifuged for 10 min at 2000 rpm and 200 μl of the supernatants are transferred to a 96 well plate and frozen at −20° C. for subsequent analysis of TNF concentration by ELISA.

Data analysis by dedicated software calculates for each compound/dose:

$$Percent inhibition of TNF α = \frac{Mean TNFα ( Controls ) - Mean TNFα ( Treated ) \times 100 }{Mean TNFα ( Controls )}$$

Test as an Anti-arthritic Agent

Activity of a compound as an anti-arthritic is tested in the collagen-induced arthritis (CIA) as defined by D. E. Trentham et al., (1977) J. Exp. Med. 146,:857. In this model acid soluble native type II collagen causes polyarthritis in rats when administered in Freunds incomplete adjuvant. Similar conditions can be used to induce arthritis in mice and primates.

Test as an Anti-cancer Agent

Activity of a compound as an anti-cancer agent may be assessed essentially as described in I. J. Fidler (1978) Methods in Cancer Research 15:399-439, using for example the B16 cell line (described in B. Hibner et al., Abstract 283 p75 10th NCI-EORTC Symposium, Amsterdam Jun. 16-19, 1998).

The invention will now be illustrated but not limited by the following Example:

### EXAMPLE 1

### N-(4-fluorophenyl)-N′-(2-benzyl-3-mercaptopropane-1-sulphonyl)-piperazine

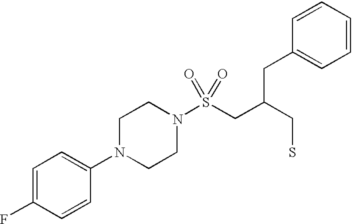

N-(4-fluorophenyl)-N′-(2-benzyl-3-acetylmercaptopropane-1-sulphonyl)-piperazine (500 mg) was added to a solution of 2M sodium hydroxide (1.1 ml) in THF (5 ml) and methanol (10 ml) and stirred for 1 hour. The reaction mixture was concentrated and the residue was partitioned between ethyl acetate (10 ml) and water (10 ml) and acetic acid (0.25 ml). The ethyl acetate fraction was collected and dried and the residue obtained on evaporation was chromatographed on a Bond-elut column eluting with a solvent gradient of hexane to 30% ethyl acetate in hexane mixture to give N-(4-fluorophenyl)-N′-(2-benzyl-3-mercaptopropane-1-sulphonyl)-piperazine (340 mg) as a white solid after tritration with diethyl ether, M Pt 73 ° C. ¹H Nmr (CDCl₃): 2.6-2.7 (m, 2H), 2.8-2.95 (m, 4H), 3.05-3.24 (m, 5H), 3.28 (m, 4H), 6.8-7.05 (m, 4H), 7.1-7.4 (m, 5H). M+H 408.

N-(4-fluorophenyl)-N′-(2-benzyl-3-acetylmercaptopropane-1-sulphonyl)-piperazine:

Thiolacetic acid (0.59 g) was added to a suspension of sodium hydride (0.52 g of a 60% dispersion in mineral oil) in DMF (25 ml) at 0° C. and the mixture was allowed to warm to room temperature. N-(4-fluorophenyl)-N′-(2-benzyl-3-methanesulphonyloxypropane-1-sulphonyl)-piperazine (1.7 g) was added and the mixture was heated at 100° C. for 5 hours. The reaction mixture ,as allowed to cool and the solvent evaporated. The residue obtained was dissolved in methylene chloride (20 ml) and passed through a 50 g Bond-Elute column eluting with a solvent gradient starting with isohexane and then increasing to an ethyl acetate/isohexane mixture (1:1). There was obtained N-(4-fluorophenyl)-N′-(2-benzyl-3-acetylmercaptopropane-1-sulphonyl)-piperazine as a gum, yield 1.6 g. ¹H Nmr (CDCl₃): 2.6 (m, 1H), 2.8-3.0 (m, 3H), 3.05 (m, 5H), 3.25 (m, 4H), 6.83-7.0 (m, 4H), 7.2-7.4 (m, 5H). M+H 451

N-(4-fluorophenyl)-N′-(2-benzyl-3-methanesulphonyloxypropane-1-sulphonyl)-piperazine:

Methenesulphonyl chloride (0.319 g) was added to a solution of N-(4-fluorophenyl)-N′-(2-benzyl-3-hydroxypropane-1-sulphonyl)-piperazine (1 g) and triethylamine (0.309 g) in methylene chloride (20 ml) at) ° C. The reaction mixture was stirred for 14 hours, washed with water and dried. Removal of the solvent gave the title compound, yield 1.1 g ¹H Nmr (CDCl₃): 2.7 (m, 1H), 2.9 (m, 3H), 3.05 (s, 3H), 3.1 (m, 4H), 3.3-3.4 (m, 4H), 4.2-4.3 (m, 1H), 4.4-4.5 (m, 1H). M+H 471

N-(4-fluorophenyl)-N′-(2-benzyl-3-hydroxypropane-1-sulphonyl)-piperazine:

Lithium borohydride (52 mg) was added to a solution of ethyl 2-[N-(4-fluorophenyl)piperazin-1-ylsulphonyl]-1-benzylpropionate (1 g) in THF (50 ml) at ambient temperature and was stirred for 4 hours. Additional lithium borohydride (52 mg) was added and stirring continued for 14 hours. The solvent was evaporated and the residue obtained was dissolved in dichloromethane (25 ml) and washed with water (2×20 ml), dried and evaporated. The residue obtained on removal of the solvent was purified by chromatography on a Bond-Elute column eluting with a solvent gradient, isohexane to ethyl acetate/isohexane (1:1) to give the title compound, yield , 0.4 g, MPt 118° C. ¹H Nmr (CDCl₃): 2.5 (m, 1H), 2.8-2.95 (m, 3H), 3.1 (m, 5H), 3.3 (m, 4H), 3.6-3.75 (m, 2H), 3.8-3.95 (m, 2H), 6.8-7.0 (m, 4H), 7.18-7.4 (m, 5H). M+H 393.

Ethyl 3-[N-(4-fluorophenyl)piperazin-1-ylsulphonyl]-2-benzylpropionate:

A mixture of N-(4-fluorophenyl)-piperazine (9.01 g) and triethylamine (7.0 ml) in methylene chloride (150 ml) was added dropwise to a cooled (−15° C.) solution of 2-ethoxycarbonyl-3-phenylpropanesulphonyl chloride (15.0 g) in methylene chloride (75 ml) at such a rate that the internal temperature did not exceed −5° C. The mixture was stirred for 15 minutes and quenched with dilute HCl (15 ml of 1.5M), washed with water (2×100 ml) and brine (50 ml)>. The aqueous extracts were washed with methylene chloride (100 ml) and the combined organic extracts were dried. The residue obtained on removal of the solvent was purified by chromatography on silica eluting with a mixture of ethyl acetate and isohexane (1:5) to give the title compound, yield 12.02 g, M+H=435 (434). ¹H Nmr (CDCl₃): 1.2 (t, 3H), 2.85-3.0 (m, 2H), 3.0-3.2 (b, 5H), 3.25 (b, 1H), 3.35 (b, 2H), 3.5 (dd, 1H), 4.15 (q, 2H), 6.85 (b, 2H), 7.0 (b, 2H), 7.15-7.9 (m, 5H). M+H 435.

2-Ethoxycarbonyl-3-phenylpropanesulphonyl Chloride:

Chlorine gas was bubbled into a suspension of ethyl-2-(acetylthiomethyl)-3-phenylpropionate (16 g) until the reaction mixture became yellow. The reaction mixture was purged with nitrogen and the mixture was concentrated under reduced pressure. The residue was extracted with methylene chloride (2×200 ml) washed with brine (50 ml) and dried to give the title compound as a yellow oil, yield 15.0 g which was used without further purification. ¹H Nmr (CDCl₃): 1.2 (t, 3H), 2.95 (dd, 1H), 3.2 (dd, 1H), 3.45 (q, 1H), 3.65 (dd, 1H), 4.2 (m, 3H), 7.1-7.4 (5H).

Ethyl-2-(acetylthiomethyl)-3-phenylpropionate:

A mixture of ethyl 2-benzylacrylate (CAS No. 20593-63-9) (20 g) and thiolacetic acid (14.2 g) was heated at 70° C. for 14 hours. The mixture was concentrated under reduced pressure and the residue was passed through silica (50 g) eluting with an ethyl acetate/isohexane mixture (1:9) to give the title compound as a yellow oil, yield 31 g. ¹H Nmr (CDCl₃): 1.15 (t, 3H), 2.3 (s, 3H), 2.8-3.2 (m, 5H), 4.1 (q, 2H), 7.1-7.3 (m, 5H).

What we claim is:

1. A compound of the formula I or a pharmaceutically acceptable salt or an in vivo hydrolysable ester thereof 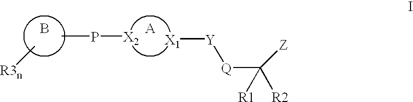
wherein ring B is a monocyclic or bicyclic alkyl, aryl, aralkyl, heteroaryl or heteroaralkyl ring comprising up to 12 ring atoms and containing one or more heteroatoms independently chosen from N, O, and S; alternatively ring B may be biphenyl; ring B may optionally be linked to ring A by a C1-4 alkyl or a C1-4 alkoxy chain linking the 2-position of ring B with a carbon atom alpha to X2;
each R3 is independently selected from hydrogen, halogen, NO2, COOR wherein R is hydrogen or C1-6alkyl, CN, CF3, C1-6 alkyl, —S—C1-6 alkyl, —SO—C1-6 alkyl, —SO2-C1-6 alkyl, C1-6 alkoxy and up to C10 aryloxy, n is 1,2 or 3;
P is —(CH₂)n- wherein n=0, 1, 2: or P is an alkene or alkyne chain of up to six carbon atoms; where X2 is C; P may be -Het-, —(CH[R6])n-Het-, -Het-(CH[R6]n-or -Het-(CH[R6])n-Het-, wherein Het is selected from —CO—, —S—, SO—, —SO2-, —NR6-, or —O— wherein n is 1 or 2; or P may be selected from —CO—N(R6)-, —N(R6)-CO—, —SO2-N(R6)- and —N(R6)-SO2-, and R6 is hydrogen, C1-6 alkyl, up to CI0 aralkyl or up to C9 heteroalkyl;
ring A is a 5-7 membered aliphatic ring and may optionally be mono- or di-substituted by optionally substituted C1-6 alkyl or C1-6 alkoxy, each substituent being independently selected from halogen, C1-6 alkyl or an oxo group;
X1 and X2 are independently selected from N and C, where a ring substituent on ring A is an oxo group this is preferably adjacent a ring nitrogen atom;
Y is selected from —SO2- and —CO—;
Q is selected from —C(R7)(R8)-, —C(R7)(R8)-CH2-, —N(R7)-, and —N(R7)-CH2- wherein R7 is hydrogen, C1-6 alkyl, up to C10 aralkyl, up to C9 heteroalkyl, up to C10 aryl, up to C9 heteroaryl, and R8 is H, C1-6 alkyl, or together with R7 forms a carbocyclic or heterocyclic spiro 5, 6 or 7 membered ring, the latter containing at least one heteroatom selected from N, O, and S;
R1 is H, C1-6 alkyl, C5-7 cycloalkyl, up to C10aryl, up to C10heteroaryl, up to C12aralkyl, or up to C12heteroarylalkyl, all optionally substituted by up to three groups independently selected from NO2, CF3, halogen, C1-4alkyl, carboxy(C1-4)alkyl, up to C6cycloalkyl, —OR4, -SR4, C1-4alkyl substituted with —OR4, SR4 (and its oxidised analogues), NR4, N—Y—R4, or C1-4alkyl-Y—NR4;
R4 is hydrogen, C1-6 alkyl, up to C10 aryl or up to C10 heteroaryl or up to C9 aralkyl, each independently optionally substituted by halogen, NO2, CN, CF3, C1-6 alkyl, —S—C1-6 alkyl, —SO—C1-6 alkyl, —SO2-C1-6 alkyl or C1-6 alkoxy;
R2 is H, C1-6 alkyl, or together with R1 forms a carbocyclic or heterocyclic spiro 5, 6 or 7 membered ring, the latter containing at least one heteroatom selected from N, O, and S;
also the group Q can be linked to either R1 or R2 to form a 5, 6 or 7 membered alkyl or heteroalkyl ring comprising one or more of O, S and N;
Z is CH₂SR wherein R is hydrogen or —COCH₃.

2. A compound as claimed in claim 1 or a pharmaceutically acceptable salt or an in vivo hydrolysable ester thereof wherein:
Q is selected from —CH(R7)-, —CH(R7)-CH2-, or —N(R7)-CH2-;
R7 is hydrogen or C1-6 alkyl;
Q is optionally linked to R1 or R2 to form a C5-7 alkyl or heteroalkyl ring.

3. A compound as claimed in claim 1 or a pharmaceutically acceptable salt or an in vivo hydrolysable ester thereof wherein.
ring A is a 5-6 membered aliphatic ring, optionally mono- or di-substituted by optionally substituted C1-6 alkyl or C1-6 alkoxy, each substituent being independently selected from halogen, C1-6 alkyl or an oxo group;
R3 is hydrogen, halogen, NO2, CF3, C1-4 alkyl, or C1-4 alkoxy, n is 1 or 2;
ring B is a monocyclic or bicyclic aryl, aralkyl or heteroaryl having up to 10 ring atoms;
P is —(CH2)n- wherein n is 0 or 1, or —O—, or —CO—N(R6)-;
one or both of X2 and X1 is N, or X1 is N, or X2 is C;
Q is —CH(R7)-, —CH(R7)-CH2-, or —N(R7)-CH2- wherein R7 is hydrogen or C1-6 alkyl; Q is optionally linked to R1 or R2 to form a C5-7 alkyl or heteroalkyl ring;
R1 is hydrogen, C1-6alkyl, C5-7 cycloalkyl, up to C12aralkyl, up to C11heteroarylalkyl, up to C10aryl or heteroaryl; all optionally substituted by up to three halogen atoms, or by CF3;
R2 is hydrogen, or together with R1 represent a carbocyclic or heterocyclic spiro 5- or 6 membered ring.

4. A compound as claimed in claim 1 or a pharmaceutically acceptable salt or an in vivo hydrolysable ester thereof wherein:
R3 is hydrogen, halogen, NO2, CF3, methyl, ethyl, methoxy, ethoxy;
ring B is a monocyclic aryl, aralkyl or heteroaryl ring having up to 7 ring atoms;
P is a direct bond;
both X2 and X1 are N;
Y is —SO2-;
Q is —CH2-;
R1 is phenyl, 4-trifluoromethylphenyl, phenethyl, phenpropyl, isobutyl, cyclopentyl, benzyloxymethyl, 3,4-dichlorophenyl, pyridyl, pyridylethyl, thiophenylpropyl, bromothiophenyl, pyrimidinylethyl, pyrimidinylpropyl, pyridylethyl, pyridylpropyl or together with R2 is spirocyclohexane or spiro-4-pyran;
R2 is hydrogen.

5. A compound as claimed in claim 1 or a pharmaceutically acceptable salt or an in vivo hydrolysable ester thereof wherein R3 is halogen and Q is —CH2-.

6. A compound as claimed in any one of the previous claims or a pharmaceutically acceptable salt or an in vivo hydrolysable ester thereof wherein ring A is a piperazine ring.

7. A compound as claimed in any one of the previous claims or a pharmaceutically acceptable salt or an in vivo hydrolysable ester thereof wherein ring B is a monocyclic aryl, aralkyl or heteroaryl ring having up to 7 ring atoms.

8. A compound as claimed in claim 7 or a pharmaceutically acceptable salt or an in vivo hydrolysable ester thereof wherein ring B is phenyl, biphenyl, napthyl, pyridyl, pyrimidinyl, pyrazinyl or pyridazinyl.

9. A compound as claimed in claim 1 or a pharmaceutically acceptable salt or an in vivo hydrolysable ester thereof wherein the compound of the formula I is N-(4-fluorophenyl)-N′-(2-benzyl-3-mercaptopropane-1-sulphonyl)-piperazine.

10. A pharmaceutical composition which comprises a compound of the formula I as claimed in claim 1 or a pharmaceutically acceptable salt or an in vivo hydrolysable ester thereof and a pharmaceutically acceptable carrier.

11. A compound of the formula I as claimed in claim 1 or a pharmaceutically acceptable salt or in vivo hydrolysable ester thereof for use in a method of therapeutic treatment of the human or animal body.

12. A compound of the formula I as claimed in claim 1 or a pharmaceutically acceptable salt or in vivo hydrolysable ester thereof for use as a therapeutic agent.

13. A method of treating a metalloproteinase mediated disease condition which comprises administering to a warm-blooded animal a therapeutically effective amount of a compound of the formula I or a pharmaceutically acceptable salt or in vivo hydrolysable ester thereof.

14. A method of treating a metalloproteinase mediated disease condition as claimed in claim 13 which comprises treating a disease condition mediated by one or more of the following enzymes: MMP13, aggrecanase, MMP9, MMP12.

15. The use of a compound of the formula I or a pharmaceutically acceptable salt or in vivo hydrolysable precursor thereof in the preparation of a medicament for use in the treatment of a disease condition mediated by one or more metalloproteinase enzymes.

16. The use of a compound of the formula I or a pharmaceutically acceptable salt or in vivo hydrolysable precursor thereof in the preparation of a medicament for use in the treatment of arthritis.

17. The use of a compound of the formula I or a pharmaceutically acceptable salt or in vivo hydrolysable precursor thereof in the preparation of a medicament for use in the treatment of atherosclerosis.

18. The use of a compound of the formula I or a pharmaceutically acceptable salt or in vivo hydrolysable precursor thereof in the preparation of a medicament for use in the treatment of chronic obstructive pulmonary diseases.

19. A process for preparing a compound of the formula I or a pharmaceutically acceptable salt or in vivo hydrolysable ester thereof which process comprises
(a) reacting a compound of the formula II with a compound of the formula III 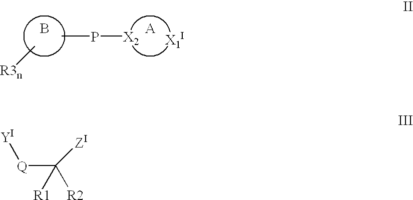
wherein X₁^{l} represents X₁ or a precursor of X₁ (whether by modification or displacement) or an activated form of X₁ suitable for reaction with Y^{l};
Y^{l} represents Y, a precursor of Y, or an activated form of Y suitable for reaction with X₁^{l};
Z^{l} represents an acid or ester group or a protected aldehyde, following reaction of II and III this is converted to a group —CH₂X wherein X represents a leaving group, this in turn is reacted with an appropriate sulphur reagent to yield the group Z; and optionally thereafter forming a pharmaceutically acceptable salt or in vivo hydrolysable ester of the compound of formula I;
or
b) reacting a compound of the formula IV with a compound of the formula V 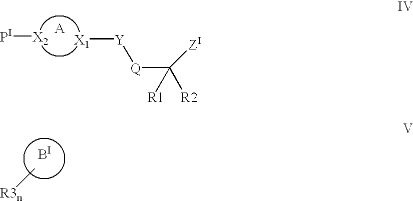
wherein B^{l} represents a suitable ring function or substituent group for reaction with P^{l};
Z^{l} is a protected thiol group; and
P^{l} represents a suitably activated form of the linker P for reaction with B^{l} or where X2 is N then P^{l} may be present on ring A rather than ring B or, as required, the linker P may be formed by appropriate reaction of precursor groups P″ and P′″ provided on rings B^{l} and A respectively, or vice versa;
and deprotecting the group Z^{l} to yield the group Z;
and optionally thereafter forming a pharmaceutically acceptable salt or in vivo hydrolysable ester of the compound of formula I.
